(19) United States
(12) Patent Application Publication
(10) Pub. No.: US 2006/0177397 A1
(43) Pub. Date: Aug. 10, 2006

(54) Self-tanning product comprising two components and means for artificially coloring the skin

(75) Inventor: Didier Candau, Bievres (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, PARIS (FR)

(21) Appl. No.: 11/311,691

(22) Filed: Dec. 20, 2005

Related U.S. Application Data
(60) Provisional application No. 60/646,596, filed on Jan. 26, 2005.

(30) Foreign Application Priority Data
Dec. 20, 2004 (FR) FR 04/53080

(51) Int. Cl. A61K 8/42

(52) U.S. Cl. 424/59

(57) ABSTRACT

Self-tanning products for the skin, including compartmentalized kits therefor, comprise at least two components (A) and (B) comprising: a first component (A) containing, formulated into a cosmetically acceptable medium, at least one self-tanning agent, and
a second component (B) containing, also formulated into a cosmetically acceptable medium, at least one hydroxyalkylurea of formula (I) 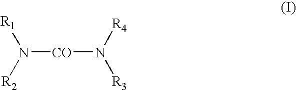
in which R₁, R₂, R₃ and R₄ each independently represent a hydrogen atom, a C₁-C₄ alkyl radical or a C₂-C₆ hydroxyalkyl radical containing from 1 to 5 hydroxyl groups, at least one of the radicals R₁-R₄ representing a hydroxyalkyl group, or salt, solvate, or isomer thereof; the components (A) and (B) being present in the same composition or confined separately.

### CROSS-REFERENCE TO PRIORITY/PROVISIONAL APPLICATIONS

This application claims priority under 35 U.S.C. § 119 of FR 04/53080, filed Dec. 20, 2004, and of provisional application Ser. No. 60/646,596, filed Jan. 26, 2005, each hereby expressly incorporated by reference and each assigned to the assignee hereof.

### CROSS-REFERENCE TO COMPANION APPLICATIONS

Copending applications Ser. No. ______ [Attorney Docket No. 016800-731], Ser. No. ______ [Attorney Docket No. 016800-732], Ser. No. ______ [Attorney Docket No. 016800-733], Ser. No. ______ [Attorney Docket No. 016800-734] and Ser. No. ______ [Attorney Docket No. 016800-735], each filed concurrently herewith, each hereby expressly incorporated by reference and each assigned to the assignee hereof.

### BACKGROUND OF THE INVENTION

1. Technical Field of the Invention

The present invention relates to a self-tanning product for the skin, comprising at least two components (A) and (B) which comprise:

a first component (A) containing, formulated into a cosmetically acceptable medium, at least one self-tanning agent, and

a second component (B) containing, also formulated into a cosmetically acceptable medium, at least one hydroxyalkylurea of formula (I); the components (A) and (B) being part of the same composition or else packaged separately.

The present invention also relates to various devices comprising several compartments, also called “kit” or “set for coloring the skin”, comprising:

a first compartment which comprises the component (A) containing the self-tanning agent(s), and

a second compartment which comprises the component (B) containing the hydroxyalkylurea compound.

The present invention also relates to a cosmetic treatment regime or regimen for artificially tanning or browning the skin, employing this self-tanning agent.

For the purpose of the present application, the term “self-tanning agent” means an agent which, when applied to the skin, in particular to the face, makes it possible to obtain a tanning effect that is more or less similar in appearance to that that can result from prolonged exposure to the sun (natural tanning) or under a UV-lamp.

2. Description of Background and/or Related and/or Prior Art

Today, it is important to look well, and a tanned skin is always a sign of good health. However, natural tanning is not always desirable in so far as it requires prolonged exposure to UV radiation, in particular to UV-A radiation that causes tanning of the skin but, in return, may induce reactions or even an alteration thereof, in particular in the case of sensitive skin or of skin continually exposed to solar radiation: erythema, burning, loss of elasticity, appearance of wrinkles, premature aging. It is therefore desirable to find an alternative to natural tanning that is compatible with the requirements of such skin.

Most of the cosmetic products intended for artificial tanning of the skin are based on carbonylated derivatives that, by interaction with the amino acids and/or the peptides of the skin (due essentially to a Maillard-type reaction) allow the formation of colored products, among which are mono- or polycarbonylated compounds such as, for example, isatin, alloxane, ninhydrin, glyceraldehyde, mesotartaric aldehyde, glutaraldehyde, erythrulose or dihydroxyacetone (DHA).

DHA is a particularly advantageous compound that is commonly used in cosmetics as an agent for artificially tanning the skin; when applied to the latter, in particular to the face, it makes it possible to obtain a tanning or browning effect that is similar in appearance to that that can result from prolonged exposure to the sun (natural tanning) or under a UV-lamp.

A drawback of DHA is the slowness with which the coloration develops: several hours (3 to 5 hours in general) are in fact needed for the coloration to be revealed. The intensity of the coloration obtained on the skin and/or its fastness over time (in particular resistance to washing) and/or the rapidity with which the coloration develops, and/or its homogeneity are often judged to be insufficient by users of DHA-based self-tanning compositions.

There exists therefore an increasing demand for self-tanning products that act rapidly and provide a coloration similar to natural tanning.

### SUMMARY OF THE INVENTION

Surprisingly and advantageously, it has now been found that the various drawbacks indicated above can be ameliorated or remedied using an agent that is a self-tanning product for the skin comprising at least two components stored or packaged separately, which comprises a first component (A) containing at least one self-tanning agent and a second component (B) containing at least one hydroxyalkylurea of formula (I). Such an arrangement makes it possible to improve the coloration of compositions comprising a self-tanning agent, in particular in terms of the rapidity of development of the color after application, the intensity of the color, the homogeneity on the skin, and the stability of the color obtained.

The present invention therefore features a self-tanning product for the skin comprising at least two components stored separately, which comprises at least:

a first component (A) containing, formulated into a cosmetically acceptable medium, at least one self-tanning agent, and

a second component (B) containing, also formulated into a cosmetically acceptable medium, at least one hydroxyalkylurea of formula (I); the components (A) and (B) being part of the same composition or else stored separately.

The present invention also features various devices comprising several compartments, also called “kit” or “set for coloring the skin”, comprising:

a first compartment which comprises the component (A) containing the self-tanning agent(s), and

a second compartment which comprises the component (B) containing the hydroxyalkylurea.

The present invention also features a cosmetic treatment regime or regimen for artificially tanning or browning the skin, wherein the component (A) and the component (B) are applied to the skin either simultaneously in the same composition, or successively or spread out over time.

In accordance with the method of the invention, when the application is carried out in two steps, the component (A) can be applied before or after the application of the component (B).

The present invention also features the use of at least one hydroxylalkylurea of formula (I) as defined below, in a self-tanning product for the skin comprising at least two components, a first component (A) containing at least one self-tanning agent and a second component (B) containing at least one hydroxyalkylurea, for improving the coloration of said self-tanning agent.

Other characteristics, aspects and advantages of the present invention will become apparent from reading the detailed description that will follow.

### DETAILED DESCRIPTION OF BEST MODE AND SPECIFIC/PREFERRED EMBODIMENTS OF THE INVENTION

The hydroxyalkylureas in accordance with the invention are selected from among those corresponding to general formula (I): 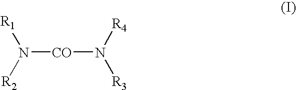
in which R₁, R₂, R₃ and R₄ each independently represent a hydrogen atom, a C₁-C₄ alkyl radical or a C₂-C₆ hydroxyalkyl radical containing from 1 to 5 hydroxyl groups, at least one of the radicals R₁-R₄ representing a hydroxyalkyl group, and also the salts, solvates and isomers thereof.

In formula (I), among the alkyl radicals, mention may in particular be made of methyl, ethyl, n-propyl, isopropyl, n-butyl, isobutyl and tert-butyl radicals.

The compounds of formula (I) that are preferred are those that contain only one hydroxyalkyl group, i.e., those for which R₁ is a hydroxyalkyl group and R₂, R₃ and R₄ represent, independently of one another, a hydrogen atom or a C₁-C₄ alkyl radical. The compounds of formula (I) for which R₁ is a hydroxyalkyl group and R₂, R₃ and R₄ each represent a hydrogen atom are more particularly preferred.

Among the hydroxyalkyl groups, preference is given to those containing a single hydroxyl group, and in particular hydroxyethyl, hydroxypropyl, hydroxybutyl, hydroxypentyl and hydroxyhexyl groups. The hydroxyethyl group is preferred.

As compounds of formula (I) that are preferred, mention may be made of N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(trishydroxymethylmethyl)urea; N-ethyl-N′-(2-hydroxyethyl)urea; N,N-bis-(2-hydroxyethyl)urea; N,N′-bis-(2-hydroxyethyl)urea; N,N-bis-(2-hydroxypropyl)urea; N,N′-bis-(2-hydroxypropyl)urea; N,N-bis-(2-hydroxyethyl)-N′-propylurea; N,N-bis-(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis-(2-hydroxyethyl)-N′,N′-dimethylurea; N,N, N′,N′-tetrakis-(2-hydroxyethyl)urea; and N′,N′-bis-(2-hydroxyethyl)-N′,N′-bis-(2-hydroxypropyl)urea.

A compound that is particularly preferred for use in the present invention is N-(2-hydroxyethyl)urea, hereinafter referred to as “hydroxyethylurea”.

The hydroxyalkylureas of formula (I) can be prepared as described in DE-2703185. Among these, hydroxyethylurea is also commercially available, in the form of a mixture at 50% by weight in water, from the company National Starch under the trademark Hydrovance®.

Among the salts, mention may be made of salts of inorganic acids, such as sulfuric acid, hydrochloric acid, hydrobromic acid, hydriodic acid, phosphoric acid or boric acid. Mention may also be made of the salts of organic acids, which may contain one or more carboxylic, sulfonic or phosphonic acid groups. They may be linear, branched or cyclic aliphatic acids or else aromatic acids. These acids may also contain one or more hetero atoms selected from O and N, for example in the form of hydroxyl groups. Mention may in particular be made of propionic acid, acetic acid, terephthalic acid, citric acid and tartaric acid.

The term “solvate” means a stoichiometric mixture of said compound of formula (I) with one or more molecules of water or of organic solvent, such a mixture being derived from the synthesis of the compound of formula (I).

When the components (A) and (B) are in the same composition, the hydroxyalkylurea(s) in accordance with the invention is (are) preferably present at contents of from 0.01 to 50% by weight, and more preferably from 0.1 to 20%, and even more preferably from 0.1 to 10% by weight, relative to the total weight of the composition containing the components (A) and (B).

When the components (A) and (B) are stored separately, the hydroxyalkylurea(s) in accordance with the invention is (are) preferably present at contents of from 0.01 to 50% by weight, and more preferably from 0.1 to 20%, and even more preferably from 0.1 to 10% by weight, relative to the total weight of the component (B).

The self-tanning agents are generally selected from mono- or polycarbonylated compounds such as, for example, isatin, alloxane, ninhydrin, glyceraldehyde, mesotartaric aldehyde, glutaraldehyde, erythrulose, the pyrazolin-4,5-dione derivatives as described in FR-2-466,492 and WO 97/35842, dihydroxyacetone (DHA), and the 4,4-dihydroxypyrazolin-5-one derivatives as described in EP-903,342. DHA will preferably be used.

The DHA can be used in free and/or encapsulated form, for example in lipid vesicles such as liposomes, described in particular in WO 97/25970.

When the components (A) and (B) are in the same composition, the self-tanning agent(s) is (are) generally present in proportions ranging from 0.1 to 15% by weight relative to the total weight of the composition, and preferably from 0.2 to 10% by weight, and more preferably from 1 to 8% by weight, relative to the total weight of the composition containing the components (A) and (B).

When the components (A) and (B) are stored separately, the self-tanning agent(s) is (are) generally present in proportions ranging from 0.1 to 15% by weight relative to the total weight of the composition, and preferably from 0.2 to 10% by weight, and more preferably from 1 to 8% by weight, relative to the total weight of the component (A).

When the self-tanning agent used is dihydroxyacetone (DHA), the components (A) and (B) will preferably be stored separately.

The compositions of the invention comprising either one and/or the other of the components (A) and (B) or of the composition containing the two components (A) and (B) are generally suitable for topical application to the skin, and generally comprise a physiologically acceptable medium, i.e., a medium that is compatible with the skin. It is a cosmetically acceptable medium, i.e., a medium that has a pleasant color, odor and feel and that does not generate any unacceptable discomfort (stinging, tautness, redness) that may dissuade the consumer from using this composition.

The compositions of the invention may be in any of the forms suitable for topical application, in particular in the form of aqueous gels, or in the form of emulsions obtained by dispersion of a fatty phase (also called oily phase) in an aqueous phase (O/W), or vice versa (W/O), or of multiple emulsions (for example, W/O/W or O/W/O or O/O/W). They can be more or less fluid and can have the appearance of a white or colored cream, of an ointment, of a milk, of a lotion, of a serum, of a paste, of a powder or of a solid stick, and can optionally be packaged in an aerosol and be in the form of a foam or of a spray. These compositions are prepared according to the usual methods.

According to a particular embodiment of the invention, the compositions of the invention may be in the form of an emulsion and then comprise at least one oily phase. The proportion of the oily phase of the emulsion can range from 1 to 80% by weight, preferably from 2 to 50% by weight, and better still from 2 to 40% by weight, relative to the total weight of the composition. The fatty substances of the oily phase, in particular the oils, and the emulsifiers and coemulsifiers optionally present, used in the composition in the form of an emulsion, are selected from those conventionally used in cosmetics or dermatology. The emulsifier and the coemulsifier, when they are present, are generally so in a proportion ranging from 0.1 to 30% by weight, preferably from 0.3 to 20% by weight, and better still from 0.5 to 15% by weight, relative to the weight of the composition. The emulsion may also contain lipid vesicles in addition to or instead of the emulsifiers and/or coemulsifiers.

The emulsions generally contain at least one emulsifier selected from amphoteric, anionic, cationic or non-ionic emulsifiers, used alone or as a mixture. The emulsifiers are selected in an appropriate manner according to the continuous phase of the emulsion to be obtained (W/O or O/W). When the emulsion is a multiple emulsion, it generally comprises an emulsifier in the primary emulsion and an emulsifier in the external phase into which the primary emulsion is introduced.

As emulsifiers that can be used for preparing the W/O emulsions, mention may, for example, be made of alkyl esters or ethers of sorbitan, of glycerol or of sugars; silicone surfactants, for instance dimethicone copolyols, such as the mixture of cyclomethicone and of dimethicone copolyol, marketed under the names DC 5225 C and DC 3225 C by Dow Corning, and alkyldimethicone copolyols such as the laurylmethicone copolyol marketed under the name “Dow Corning 5200 Formulation Aid” by Dow Corning, cetyldimethicone copolyol marketed under the name Abil EM 90® by Goldschmidt and the mixture of polyglyceryl-4 isostearate/cetyldimethicone copolyol/hexyl laurate, marketed under the name Abil WE 09® by Goldschmidt. One or more coemulsifiers may also be added thereto, which coemulsifiers may advantageously be selected from the group comprising branched-chain fatty acid esters of polyol, and especially branched-chain fatty acid esters of glycerol and/or of sorbitan, for example polyglyceryl isostearate, such as the product marketed under the name Isolan GI 34 by Goldschmidt, sorbitan isostearate, such as the product marketed under the name Arlacel 987 by ICI, and sorbitan glyceryl isostearate, such as the product marketed under the name Arlacel 986 by ICI, and mixtures thereof.

As emulsifiers that can be used for preparing the O/W emulsions, mention may, for example, be made of non-ionic emulsifiers such as oxyalkylenated (more particularly polyoxyethylenated) fatty acid esters of polyols, for example polyethylene glycol stearates, for instance PEG-100 stearate, PEG-50 stearate and PEG-40 stearate; oxyalkylenated fatty acid esters of sorbitan comprising, for example, from 20 to 100 EO, and for example those marketed under the trademarks Tween 20 or Tween 60 by Uniqema; oxyalkylenated (oxyethylenated and/or oxypropylenated) fatty alcohol ethers; alkoxylated or non-alkoxylated sugar esters, for instance sucrose stearate and PEG-20 methylglucose sesquistearate; sorbitan esters such as the sorbitan palmitate marketed under the name Span 40 by Uniqema; esters of diacid and of fatty alcohol, such as dimyristyl tartrate; mixtures of these emulsifiers, for instance the mixture of glyceryl stearate and of PEG-100 stearate (CTFA name: glycerylstearate/PEG-100 stearate) marketed under the name Arlacel 165 by Uniqema and under the name Simulsol 165 by Seppic; or the mixture of dimyristyl tartrate, of cetearyl alcohol, of Pareth-7 and of PEG-25 laureth-25, marketed under the name Cosmacol PSE by Sasol (CTFA name: dimyristyl tartrate/cetearyl alcohol/12-15 Pareth 7/PPG 25 laureth 25).

Coemulsifiers may be added to these emulsifiers, for instance fatty alcohols containing from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol, or fatty acids.

Emulsions free of emulsifying surfactants or containing less than 0.5% thereof relative to the total weight of the composition (A) or (B) may also be prepared, by using suitable compounds for stabilizing said emulsions, for example amphiphilic polymers, electrolytes.

When the composition of the invention is in emulsion form, it comprises at least one oily phase that contains at least one oil, in particular a cosmetic oil. The term “oil” means a fatty substance that is liquid at room temperature (25° C.).

As oils that can be used in the composition of the invention, use may, for example, be made of hydrocarbon-based oils of animal origin, such as perhydrosqualene (or squalane); hydrocarbon-based oils of plant origin, such as caprylic/capric acid triglycerides, for instance those marketed by Stearineries Dubois or those marketed under the names Miglyol 810, 812 and 818 by Dynamit Nobel, or alternatively oils of plant origin, for example sunflower oil, corn oil, soybean oil, marrow oil, grapeseed oil, sesame oil, hazelnut oil, apricot oil, macadamia oil, arara oil, coriander oil, castor oil, avocado oil, jojoba oil or shea butter oil; synthetic oils; silicone oils, for instance volatile or non-volatile polymethylsiloxanes (PDMS) containing a linear or cyclic silicone chain, which are liquid or pasty at ambient temperature; fluoro oils, such as partially hydrocarbon-based and/or partially silicone-based fluoro oils, for instance those described in document JP-A-2-295912; ethers such as dicaprylyl ether (CTFA name: dicaprylyl ether); and C₁₂-C₁₅ fatty alcohol benzoates (Finsolv TN from Finetex); arylalkyl benzoate derivatives such as 2-phenylethyl benzoate (X-Tend 226 from ISP); amidated oils such as isopropyl N-lauroylsarcosinate (Eldew SL-205 from Ajimoto), and mixtures thereof.

The compositions of the invention may contain one or more organic solvents which may be selected from the group consisting of hydrophilic organic solvents, lipophilic organic solvents, amphiphilic solvents, or mixtures thereof.

Among the hydrophilic organic solvents, mention may, for example, be made of linear or branched monohydric alcohols containing from 1 to 8 carbon atoms, such as ethanol, propanol, butanol, isopropanol or isobutanol; polyethylene glycols containing from 6 to 80 ethylene oxides; polyols such as propylene glycol, isoprene glycol, butylene glycol, glycerol or sorbitol; monoalkyl or dialkyl isosorbide in which the alkyl groups contain from 1 to 5 carbon atoms, such as dimethyl isosorbide; glycol ethers, such as diethylene glycol monomethyl ether or diethylene glycol monoethyl ether, and propylene glycol ethers such as dipropylene glycol methyl ether.

As amphiphilic organic solvents, mention may be made of polypropylene glycol (PPG) derivatives, such as esters of polypropylene glycol and of a fatty acid, or of PPG and of a fatty alcohol, for instance PPG-23 oleyl ether and PPG-36 oleate.

As lipophilic organic solvents, mention may, for example, be made of fatty esters such as diisopropyl adipate, dioctyl adipate or alkyl benzoates.

The compositions in accordance with the present invention may also comprise conventional cosmetic adjuvants selected from softeners, humectants, opacifiers, stabilizers, emollients, silicones, anti-foams, fragrances, preservatives, anionic, cationic, non-ionic, zwitterionic or amphoteric surfactants, fillers, polymers, propellants, basifying or acidifying agents, or any other ingredient normally used in cosmetics and/or dermatology.

As hydrophilic thickeners, mention may be made of carboxyvinyl polymers such as carbopols (carbomers) and Pemulen (acrylate/C₁₀-C₃₀ alkyl acrylate copolymer); cellulose derivatives such as hydroxyethylcellulose; polysaccharides, and in particular gums such as xanthan gum; and mixtures thereof.

As lipophilic thickeners, mention may be made of modified clays such as hectorite and derivatives thereof, for instance the products marketed under the name Bentone.

As preservatives, mention may be made of para-hydroxybenzoic acid esters, also called Parabens® (in particular methyl paraben, ethyl paraben, propyl paraben), phenoxyethanol, compounds which release formaldehyde, such as, for example, imidazolidinylurea or diazolidinylurea, chlorhexidine digluconate, sodium benzoate, caprylyl glycol, iodopropynyl butyl carbamate, pentylene glycol, alkyltrimethylammonium bromide, such as myristyltrimethylammonium bromide (CTFA name: myrtrimonium bromide), dodecyltrimethylammonium bromide, hexadecyltrimethylammonium bromide, and mixtures thereof, such as the mixture marketed under the name Cetrimide® by FEF Chemicals. The preserving agent may be present in the composition according to the invention at a content ranging from 0.001 to 10% by weight, relative to the total weight of the composition, especially ranging from 0.1 to 5% by weight, and in particular ranging from 0.2 to 3% by weight.

The components (A) and/or (B) in accordance with the present invention can also comprise one or more additional coloring agents.

For the purpose of the present invention, the expression “agent for coloring the skin” means any compound that has a particular affinity for the skin, allowing it to confer on the latter a long-lasting, non-covering coloration (i.e., that does not have a tendency to opacify the skin) that is not removed either with water or with a solvent, and that is resistant both to rubbing and to washing with a solution containing surfactants. Such a long-lasting coloration therefore differs from the superficial and momentary coloration provided, for example, by a makeup pigment.

The additional coloring agents can also be selected, for example, from other plant extracts, for instance extracts of sorghum obtained from the whole plant, the stems, the seeds or the leaves of the Sorghum genus. The preferred Sorghum species are selected from Sorghum bicolor, Sorghum caudatum, Sorghum nervosum, Sorghum durra, Sorghum vulgare and Sorghums in combination with Colletotrichum graminicola such as those described in FR-0-200,251.

The additional coloring agents may also be iron oxide pigments, of which the mean size of the elemental particles is less than 100 nm, such as those described in EP-966,953.

The components (A) and/or (B) in accordance with the present invention may also comprise dyes for coloring the compositions of the invention so as to facilitate the pinpointing of the area of the skin to be colored and/or to modify the color produced by the self-tanning agent.

These dyes may be selected from synthetic or natural direct dyes.

These dyes may be selected, for example, from red or orange dyes of the fluorane type, such as those described in FR-2-840,806. Mention may, for example, be made of the following dyes:

tetrabromofluoroscein or eosin known under the CTFA name: CI 45380 or Red 21;

phloxine B known under the CTFA name: CI 45410 or Red 27;

diiodofluorescein known under the CTFA name: CI 45425 or Orange 10;

dibromofluorescein known under the CTFA name: CI 45370 or Orange 5;

tetrabromofluoroscein sodium salt known under the CTFA name: CI 45380 (Na salt) or Red 22;

phloxine B sodium salt known under the CTFA name: CI 45410 (Na salt) or Red 28;

diiodofluorescein sodium salt known under the CTFA name: CI 45425 (Na salt) or Orange 11;

erythrosine known under the CTFA name: CI 45430 or Acid Red 51;

phloxine known under the CTFA name: CI 45405 or Acid Red 98.

These dyes may also be selected from anthraquinones, caramel, carmin, carbon black, azulene blues, methoxalene, trioxalene, guajazulene, chamuzulene, rose bengal, cosine 10B, cyanosine and daphinine.

These dyes may also be selected from indole derivatives such as the monohydroxyindoles as described in FR-2-651,126 (i.e., 4-, 5-, 6- or 7-hydroxyindole) or the dihydroxyindoles as described in EP-B-O-425,324 (i.e., 5,6-dihydroxyindole, 2-methyl-5,6-dihydroxyindole, 3-methyl-5,6-dihydroxyindole, 2,3-dimethyl-5,6-dihydroxyindole).

The components (A) and/or (B) in accordance with the invention will preferably comprise other supplementary organic or inorganic photoprotective agents that are active in the UV-A range and/or in the UV-B range and that are water-soluble or liposoluble or even insoluble in the cosmetic solvents commonly used.

The supplementary organic photoprotective agents are in particular selected from anthranilates; dibenzoylmethane derivatives; cinnamic derivatives; salicylic derivatives; camphor derivatives; benzophenone derivatives; β,β-diphenylacrylate derivatives; triazine derivatives; benzotriazole derivatives; benzylmalonate derivatives, in particular those mentioned in U.S. Pat. No. 5,624,663; benzimidazole derivatives; imidazolines; bis-benzoazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2303549, DE-19726184 and EP-893,119; benzoxazole derivatives as described in EP-O-832,642, EP-1-027,883, EP-1-300,137 and DE-10162844; screening polymers and screening silicones such as those described in particular in WO 93/04665; dimers derived from α-alkylstyrene, such as those described in DE-19855649; 4,4-diarylbutadienes as described in EP-O-967,200, DE-19746654, DE-19755649, EP-A-1-008,586, EP-1-133,980 and EP-133,981, and mixtures thereof.

As examples of supplementary organic photoprotective agents, mention may be made of those denoted below under their INCI name:

Para-Aminobenzoic Acid Derivatives:

PABA,

Ethyl PABA,

Ethyl dihydroxypropyl PABA,

Ethylhexyl dimethyl PABA marketed in particular under the name “Escalol 507” by ISP,

Glyceryl PABA,

PEG-25 PABA marketed under the name “Uvinul P25” by BASF.

Dibenzoylmethane Derivatives:

Butyl methoxydibenzoylmethane marketed in particular under the trademark “Parsol 1789” by Hoffmann Laroche,

Isopropyldibenzoylmethane.

Salicylic Derivatives:

Homosalate marketed under the name “Eusolex HMS” by Rona/EM Industries,

Ethylhexyl salicylate marketed under the name “Neo Heliopan OS” by Haarmann and Reimer,

Dipropylene glycol salicylate marketed under the name “Dipsal” by Scher, TEA salicylate marketed under the name “Neo Heliopan TS” by Haarmann and Reimer.

Cinnamic Derivatives:

Ethylhexyl methoxycinnamate marketed in particular under the trademark “Parsol MCX” by Hoffmann La Roche,

Isopropyl methoxycinnamate,

Isoamyl methoxycinnamate marketed under the trademark “Neo Heliopan E 1000” by Haarmann and Reimer,

Cinoxate,

DEA methoxycinnamate,

Diisopropyl methylcinnamate,

Glyceryl ethylhexanoate dimethoxycinnamate.

β,β-Diphenylacrylate Derivatives:

Octocrylene marketed in particular under the trademark “Uvinul N539” by BASF,

Etocrylene marketed in particular under the trademark “Uvinul N35” by BASF.

Benzophenone Derivatives:

Benzophenone-1 marketed under the trademark “Uvinul 400” by BASF,

Benzophenone-2 marketed under the trademark “Uvinul D50” by BASF,

Benzophenone-3 or oxybenzone, marketed under the trademark “Uvinul M40” by BASF,

Benzophenone-4 marketed under the trademark “Uvinul MS40” by BASF,

Benzophenone-5,

Benzophenone-6 marketed under the trademark “Helisorb 11” by Norquay,

Benzophenone-8 marketed under the trademark “Spectra-Sorb UV-24” by American Cyanamid,

Benzophenone-9 marketed under the trademark “Uvinul DS-49” by BASF, Benzophenone-12,

n-Hexyl 2-(4-diethylamino-2-hydroxybenzoyl)benzoate.

Benzylidenecamphor Derivatives:

3-Benzylidenecamphor manufactured under the name “Mexoryl SD” by Chimex,

4-Methylbenzylidenecamphor marketed under the name “Eusolex 6300” by Merck,

Benzylidenecamphorsulfonic acid manufactured under the name “Mexoryl SL” by Chimex,

Camphor benzalkonium methosulfate manufactured under the name “Mexoryl SO” by Chimex,

Terephthalylidenedicamphorsulfonic acid manufactured under the name “Mexoryl SX” by Chimex,

Polyacrylamidomethylbenzylidenecamphor manufactured under the name “Mexoryl SW” by Chimex.

Phenylbenzimidazole Derivatives:

Phenylbenzimidazolesulfonic acid marketed in particular under the trademark “Eusolex 232” by Merck,

Disodium phenyl dibenzimidazole tetrasulfonate marketed under the trademark “Neo Heliopan AP” by Haarmann and Reimer.

Phenylbenzotriazole Derivatives:

Drometrizole trisiloxane marketed under the name “Silatrizole” by Rhodia Chimie,

Methylenebisbenzotriazolyltetramethylbutylphenol, marketed in solid form under the trademark “Mixxim BB/100” by Fairmount Chemical or in micronized form in aqueous dispersion under the trademark “Tinosorb M” by Ciba Specialty Chemicals).

Triazine Derivatives:

Bis-ethylhexyloxyphenol methoxyphenyl triazine marketed under the trademark “Tinosorb S” by Ciba Geigy,

Ethylhexyltriazone marketed in particular under the trademark “Uvinul T150” by BASF,

Diethylhexylbutamidotriazone marketed under the trademark “Uvasorb HEB” by Sigma 3V,

2,4,6-tris(dineopentyl 4′-aminobenzalmalonate)-s-triazine,

2,4,6-tris(diisobutyl 4′-aminobenzalmalonate)-s-triazine.

Anthranilic Derivatives:

Menthyl anthranilate marketed under the trademark “Neo Heliopan MA” by Haarmann and Reimer.

Imidazoline Derivatives:

Ethylhexyldimethoxybenzylidenedioxoimidazoline propionate.

Benzalmalonate Derivatives:

Dineopentyl 4′-methoxybenzalmalonate,

Polyorganosiloxane containing benzalmalonate functions, such as Polysilicone-15 marketed under the trademark “Parsol SLX” by Hoffmann La Roche.

4,4-Diarylbutadiene Derivatives:

1,1-Dicarboxy(2,2′-dimethylpropyl)-4,4′-diphenylbutadiene.

Benzoxazole Derivatives:

2,4-bis[5-1 (dimethylpropyl)benzoxazol-2-yl(4-phenyl)imino]-6-(2-ethylhexyl)imino-1,3,5-triazine marketed under the name Uvasorb K2A by Sigma 3V,

and mixtures thereof.

The preferred supplementary organic photoprotective agents are selected from among:

Ethylhexyl methoxycinnamate,

Homosalate,

Ethylhexyl salicylate,

Butyl methoxydibenzoylmethane,

Octocrylene,

Phenylbenzimidazolesulfonic acid,

Benzophenone-3,

Benzophenone-4,

Benzophenone-5,

n-Hexyl 2-(4-diethylamino-2-hydroxybenzoyl)benzoate,

4-Methylbenzylidenecamphor,

Terephthalylidenedicamphorsulfonic acid,

Disodium phenyl dibenzimidazole tetrasulfonate,

Methylenebisbenzotriazolyltetramethylbutylphenol,

Ethylhexyltriazone,

Bis-ethylhexyloxyphenol methoxyphenyl triazine,

Diethylhexylbutamidotriazone,

2,4,6-tris(dineopentyl 4′-aminobenzalmalonate)-s-triazine,

2,4,6-tris(diisobutyl 4′-aminobenzalmalonate)-s-triazine,

Drometrizole trisiloxane,

Polysilicone-15,

Dineopentyl 4′-methoxybenzalmalonate,

1,1-dicarboxy(2,2′-dimethylpropyl)-4,4-diphenylbutadiene,

2,4-bis[5-1 (dimethylpropyl)benzoxazol-2-yl(4-phenyl)imino]-6-(2-ethylhexyl)imino-1,3,5-triazine,

and mixtures thereof.

The inorganic photoprotective agents are selected from pigments (average size of the primary particles: generally from 5 nm to 100 nm, preferably from 10 nm to 50 nm) of metal oxides that are coated or uncoated, such as, for example, pigments of titanium oxide (amorphous or crystallized in rutile and/or anatase form), of iron oxide, of zinc oxide, of zirconium oxide or of cerium oxide, or mixtures thereof. Conventional coating agents are, moreover, alumina and/or aluminum stearate. Such coated or uncoated metal oxide pigments are in particular described in EP-518,772 and EP-518,773.

The additional photoprotective agents are generally present in the compositions according to the invention in proportions ranging from 0.01 to 20% by weight relative to the total weight of the composition, and preferably ranging from 0.1 to 10% by weight relative to the total weight of the composition.

When the components (A) and (B) are in the same composition or are applied in the form of a mixture, the pH of the composition or of said mixture preferably ranges from 3.5 to 6.5, and more preferably from 4 to 6.

Of course, one skilled in this art will take care to choose the optional additional compound(s) mentioned above and/or the amounts thereof in such a way that the advantageous properties intrinsically associated with the two components (A) and (B) constituting the self-tanning product in accordance with the invention are not, or are not substantially, impaired by the envisaged addition(s). Those skilled in the art will also take care to incorporate the additive(s) mentioned above appropriately into one of the two components (A) and (B) when they are stored separately.

When the components (A) and/or (B) are stored separately, they are packaged in a device comprising several compartments, also called “kit” or “set for coloring the skin”, comprising:

a first compartment which comprises the component (A) containing the self-tanning agent(s), and

a second compartment which comprises the component (B) containing the hydroxyalkylurea compound.

According to a first embodiment of a device in accordance with the invention, the components (A) and (B) can be packaged independently, each in a container delimiting at least one compartment, said container being closed by means of a closing member.

The container may be in any appropriate form. It may in particular be in the form of a bottle, a tube, a jar, a case, a box, a sachet or a carton.

The closing member may be in the form of a removable stopper, a lid, a cap, a tear-off strip or a capsule, in particular of the type comprising a body attached to the container and a cover cap articulated on the body. It may also be in the form of a member for selectively closing the container, in particular a pump, a valve or a flap valve.

The product may be contained directly in the container, or indirectly. By way of example, the product may be arranged on an impregnated carrier, in particular in the form of a wipe or of a pad, and arranged (individually or in plurality) in a box or in a sachet. Such a carrier incorporating the product is described, for example, in WO 01/03538.

The closing member may be coupled to the container by screwing. Alternatively, the coupling between the closing member and the container is done other than by screwing, in particular via a bayonet mechanism, by click-fastening, gripping, welding, bonding or by magnetic attraction. The term “click-fastening” is in particular intended to mean any system involving the crossing of a bead or cord of material by elastic deformation of a portion, in particular of the closing member, followed by return to the elastically unconstrained position of said portion after the crossing of the bead or cord.

The container may be at least partially made of thermoplastic material. By way of examples of thermoplastic materials, mention may be made of polypropylene or polyethylene.

Alternatively, the container is made of non-thermoplastic material, in particular of glass or of metal (or alloy).

The container may have rigid walls or deformable walls, in particular in the form of a tube or of a tubular bottle.

The container may comprise means for distributing or facilitating the distribution of the composition. By way of example, the container may have deformable walls so as to cause the composition to exit in response to a positive pressure inside the container, this positive pressure being caused by elastic (or non-elastic) squeezing of the walls of the container.

According to another embodiment, the components, respectively, (A) and (B) are packaged in two containers joined so as to be interdependent with respect to one another.

For example, they are contained in two containers, one held with the other via an outer packaging. In particular, each container is equipped with a distributing member, for example a pump or a valve. Preferably, this distributing member is operated manually. The pump may also have no air intake in the case where the composition must be kept away from any contact with the outside, during its storage period.

According to a first embodiment, the operating of these distributing members drives the compositions into a mixing chamber of the assembly, so as to provide mixing prior to their distribution out of this assembly formed by these two joined containers.

Alternatively, according to a first embodiment, the operating of these distributing members drives the compositions into a mixing chamber of the assembly, so as to provide mixing prior to their distribution out of this assembly formed by these two joined containers.

Alternatively, according to a second embodiment, the operating of these distributing members results in the compositions exiting without prior mixing.

A particular embodiment of the invention is a device as described in EP-1-270,444. It is a device for the simultaneous distribution of the two components (A) and (B), packaged separately in first and second sachets with flexible walls, said device comprising:

i) means for maintaining the two sachets fixed in a superposed position such that respective outlet orifices of said sachets are placed in the vicinity of one another; and

ii) means that are movable with respect to the fixing means and are capable of pressurizing the two sachets so as to force their content out through their respective outlet orifices according to a predetermined ratio.

Another particular embodiment of the invention is a device as described in EP-1-300,344. It is a device for the co-distribution of the two components (A) and (B) packaged separately, comprising:

a) a first flexible-wall container containing the component (A);

b) a second flexible-wall container, exterior to the first, and containing the component (B);

c) a plate on which the first and second containers are mounted;

d) a distributing member that is movable with respect to the plate and able to irreversibly switch the device from a first configuration in which the first and second containers are isolated from at least one distribution orifice formed by said distributing member, to a second configuration in which the first and second containers are in communication with said distribution orifice(s).

Thus, in the second configuration of the device, in response to a pressure exerted on the flexible walls of the first and second containers, which are placed adjacent to one another, the first and second products can be co-distributed.

The co-exiting of the two products so as to produce a composition, in particular a cosmetic composition, can take place via a single distribution orifice, upstream of which there may be a mixing zone in which the two products are brought into contact before being distributed in mixed form.

Another particular embodiment of the device of the invention is a double pump comprising:

a) two reservoirs, preferably housed in the same outer casing comprising, respectively, the component (A) or the component (B), preferably housed in the same outer casing;

b) two pumps, each mounted on a reservoir,

c) a single push-button that can move axially along a main axis of elongation of the outer casing, and that makes it possible to activate the simultaneous opening of the two pumps; these two pumps each being, moreover, connected by virtue of an outlet channel that opens into at least one distribution orifice.

Said distribution orifice may be a single orifice common to the two reservoirs. In this case, the products exit in the mixed form.

According to another embodiment, each outlet channel is associated with its own distribution orifice. The products contained in these reservoirs are therefore distributed out of the device, without ever having been brought into contact beforehand inside the device. This type of embodiment is in particular described in FR-2789371; U.S. Pat. No. 5,224,627; WO 97/05040.

Another particular embodiment of a device of the invention is a double aerosol comprising:

a) two reservoirs, preferably housed in the same outer casing comprising at least one propellant and, respectively, the component (A) or the component (B),

b) two aerosol valves, each mounted on a reservoir,

c) a single push-button that can move axially along a main axis of elongation of the outer casing, and that makes it possible to activate the simultaneous opening of the two pumps; these two valves each being, moreover, connected by virtue of an outlet channel that opens into at least one distribution orifice.

Said distribution orifice may be a single orifice common to the two reservoirs. In this case, the products exit in mixed form.

According to another embodiment, each outlet channel is associated with its own distribution orifice. The products contained in these reservoirs are then distributed out of the device without having been brought into contact beforehand inside the device.

Of course, depending on the device used, those skilled in the art will take care, for each of the components (A) and (B), to choose the viscosity and the Theological properties that are appropriate for correct exiting of the products out of said device.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

The self-tanning product comprising two components (A) and (B) below was prepared; the amounts are indicated as percentages by weight:

| Phase | | Amounts |
|---|---|---|
| | Composition A | |
| 1 | Polydimethylsiloxane | 1.0 |
| | Preservatives | 1.0 |
| | Mixture of glyceryl monostearate/PEG (100 EO)- | 2.0 |
| | stearate | |
| | (C₁₂/C₁₃) dialkyl tartrate | 1.0 |
| | Mixture of arachyl polyglycoside and of arachidyl, | 3.0 |
| | behenyl alcohols (15/85) | |
| | C₁₂/C₁₅ alcohol benzoate | 6.0 |
| 2 | Acrylamide/sodium acrylamido-2-methylsulfonate | 1.75 |
| | copolymer as an inverse emulsion | |
| 3 | Cyclopentadimethylsiloxane | 3.0 |
| 4 | Dihydroxyacetone | 5.0 |
| 5 | Glycerol | 5.0 |
| | EDTA | 0.1 |
| | Deionized water qs | 100 |
| | Composition B | |
| 1 | Polydimethylsiloxane | 1.0 |
| | Preservatives | 1.0 |
| | Mixture of glyceryl monostearate/PEG (100 EO)- | 2.0 |
| | stearate | |
| | (C₁₂/C₁₃) dialkyl tartrate | 1.0 |
| | Mixture of arachyl polyglycoside and of arachidyl, | 3.0 |
| | behenyl alcohols (15/85) | |
| | C₁₂/C₁₅ alcohol benzoate | 6.0 |
| 2 | Acrylamide/sodium acrylamido-2-methylsulfonate | 1.75 |
| | copolymer as an inverse emulsion | |
| 3 | Cyclopentadimethylsiloxane | 3.0 |
| | Hydroxyethylurea | 5.0 |
| 5 | Glycerol | 5.0 |
| | EDTA | 0.1 |
| | Deionized water qs | 100 |

Procedure:

Phase 1 and phase 5 are weighed out separately and are brought to around 80° C. in a water bath. Phase 1 is placed on a Moritz stirrer and phase 5 is poured in. The mixture is left to stir for 10 minutes, with moderate stirring. Phase 2 is added at around 60° C. and stirring is carried out until homogenization. Phase 3 is incorporated, the mixture is homogenized, and then phase 4 with DHA is added for composition A. The mixture is homogenized and the stirring is then stopped.

### EXAMPLE 2

The self-tanning product comprising two components (A) and (B) below is prepared; the amounts are indicated as percentages by weight:

| Phase | | Amounts |
|---|---|---|
| | Composition A | |
| 1 | Polydimethylsiloxane | 1.0 |
| | Preservatives | 1.0 |
| | Mixture of glyceryl monostearate/PEG (100 EO)- | 2.0 |
| | stearate | |
| | (C₁₂/C₁₃) dialkyl tartrate | 1.0 |
| | Mixture of arachyl polyglycoside and of arachidyl, | 3.0 |
| | behenyl alcohols (15/85) | |
| | C₁₂/C₁₅ alcohol benzoate | 6.0 |
| 2 | Acrylamide/sodium acrylamido-2-methylsulfonate | 1.75 |
| | copolymer as an inverse emulsion | |
| 3 | Cyclopentadimethylsiloxane | 3.0 |
| 4 | Dihydroxyacetone | 5.0 |
| 5 | Glycerol | 5.0 |
| | EDTA | 0.1 |
| | Deionized water qs | 100 |
| | Composition B | |
| 1 | Polydimethylsiloxane | 1.0 |
| | Preservatives | 1.0 |
| | Mixture of glyceryl monostearate/PEG (100 EO)- | 2.0 |
| | stearate | |
| | (C₁₂/C₁₃) dialkyl tartrate | 1.0 |
| | Mixture of arachyl polyglycoside and of arachidyl, | 3.0 |
| | behenyl alcohols (15/85) | |
| | C₁₂/C₁₅ alcohol benzoate | 6.0 |
| 2 | Acrylamide/sodium acrylamido-2-methylsulfonate | 1.75 |
| | copolymer as an inverse emulsion | |
| 3 | Cyclopentadimethylsiloxane | 10.0 |
| | Hydroxyethylurea | |
| 5 | Glycerol | 5.0 |
| | EDTA | 0.1 |
| | Deionized water qs | 100 |

Procedure:

Phase 1 and phase 5 are weighed out separately and are brought to around 80° C. in a water bath. Phase 1 is placed on a Moritz stirrer and phase 5 is poured in. The mixture is allowed to stir for 10 minutes, with moderate stirring. Phase 2 is added at around 60° C. and stirring is carried out until homogenization. Phase 3 is incorporated, the mixture is homogenized, and then phase 4 with DHA is added for composition A. The mixture is homogenized and the stirring is then stopped.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A self-tanning product for the skin comprising at least two components, and which comprises at least:
a first component (A) containing, formulated into a cosmetically acceptable medium, at least one self-tanning agent, and
a second component (B) containing, also formulated into a cosmetically acceptable medium, at least one hydroxyalkylurea of formula (I) 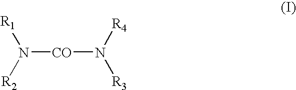
in which R₁, R₂, R₃ and R₄ each independently represent a hydrogen atom, a C₁-C₄ alkyl radical or a C₂-C₆ hydroxyalkyl radical containing from 1 to 5 hydroxyl groups, at least one of the radicals R₁-R₄ representing a hydroxyalkyl group, or salt, solvate, or isomer thereof, said components (A) and (B) being present in the same composition or confined separately.

2. The product as defined by claim 1, wherein formula (I) R₁ is a hydroxyalkyl group and R₂, R₃ and R₄ represent, independently of one another, a hydrogen atom or a C₁-C₄ alkyl radical.

3. The product as defined by claim 2, wherein formula (I) R₁ is a hydroxyalkyl group and R₂, R₃ and R₄ each represent a hydrogen atom.

4. The product as defined by claim 1, said at least one compound of formula (I) being selected from the group consisting of N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(trishydroxymethylmethyl)urea; N-ethyl-N′-(2-hydroxyethyl)urea; N,N-bis-(2-hydroxyethyl)urea; N,N′-bis-(2-hydroxyethyl)urea; N,N-bis-(2-hydroxypropyl)urea; N,N′-bis-(2-hydroxypropyl)urea; N,N-bis-(2-hydroxyethyl)-N′-propylurea; N,N-bis-(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis-(2-hydroxyethyl)-N′,N′-dimethylurea; N,N,N′,N′-tetrakis-(2-hydroxyethyl)urea; and N′,N′-bis-(2-hydroxyethyl)-N′,N′-bis-(2-hydroxypropyl)urea, and mixtures thereof.

5. The product as defined by claim 1, said at least one compound of formula (I) comprising N-(2-hydroxyethyl)urea.

6. The product as defined by claim 1, in which the components (A) and (B) are in the same composition and in which the at least one hydroxyalkylurea is present at a content of from 0.01 to 50% by weight thereof.

7. The product as defined by claim 1, in which the components (A) and (B) are packaged separately and in which the at least one hydroxyalkylurea is present at a content of from 0.01 to 50% by weight thereof.

8. The product as defined by claim 1, in which at least one the self-tanning agent comprises a mono- or polycarbonylated compound.

9. The product as defined by claim 8, said at least one self-tanning agent being selected from the group consisting of isatin, alloxane, ninhydrin, glyceraldehyde, mesotartaric aldehyde, glutaraldehyde, erythrulose, pyrazoline-4,5-dione derivatives, dihydroxyacetone (DHA), and 4,4-dihydroxypyrazoline-5-dione derivatives.

10. The product as defined by claim 9, said at least one self-tanning agent comprising DHA.

11. The product as defined by claim 1, in which the components (A) and (B) are in the same composition and in which the at least one self-tanning agent is present in a proportion ranging from 0.1 to 15% by weight thereof.

12. The product as defined by claim 1, in which the components (A) and (B) are packaged separately and in which the at least one self-tanning agent is present in a proportion ranging from 0.1 to 15% by weight thereof.

13. The product as defined by claim 1, in which the at least self-tanning agent is dihydroxyacetone (DHA) and the components (A) and (B) are packaged separately.

14. The product as defined by claim 1, in which the component (A) and/or (B) also comprises one or more additional coloring agents.

15. The product as defined by claim 1, in which the component (A) and/or (B) also comprises one or more synthetic or natural direct dyes.

16. The product as defined by claim 1, in which the component (A) and/or (B) also comprises one or more indole dyes.

17. The product as defined by claim 1, further comprising at least one organic photoprotective agent and/or at least one inorganic photoprotective agent that is active in the UV-A range and/or in the UV-B range.

18. The product as defined by claim 1, wherein the composition containing the two components (A) and (B) or the mixture of the components (A) and (B) has a pH of from 3.5 to 6.5.

19. A cosmetic regime or regimen for artificially tanning or browning the skin, comprising topically applying thereon, whether simultaneously, successively or spread out over time, the component (A) and the component (B) as defined by claim 1.

20. A procedure for enhancing the effectiveness of a self-tanning agent, comprising mixing a hydroxyalkylurea compound therewith.

21. A device comprising several compartments for coloring the skin, which comprises:
a first compartment containing the component (A) as defined by claim 1,
a second compartment containing component (B) also defined by claim 1.

22. The device as defined by claim 21, in which the components (A) and (B) are packaged independently, each in a container delimiting at least one compartment, said container being closed by means of a closing member.

23. The device as defined by claim 21, in which the components (A) and (B) are packaged in two containers joined so as to be interdependent with respect to one another.

24. The device as defined by claim 23, in which the components (A) and (B) are contained in two containers, one secured with the other via an outer packaging.

25. The device as defined by claim 24, in which each container is equipped with a distributing member.

26. The device as defined by claim 25, in which the distributing member comprises a pump or an aerosol valve.

27. The device as defined by claim 25, in which the operating of the distributing members drives the components (A) and (B) into a mixing chamber of the assembly, to provide mixing prior to their distribution out of this assembly formed by these two joined containers.

28. The device as defined by claim 25, in which the operating of the distributing members results in the components (A) and (B) exiting without prior mixing.

29. The device as defined by claim 27, wherein the two components (A) and (B) are packaged separately in first and second sachets with flexible walls, said device comprising:
i) means for maintaining the two sachets fixed in a superposed position such that respective outlet orifices of said sachets are placed in the vicinity of one another; and
ii) means that are movable with respect to the fixing means and are capable of pressurizing the two sachets so as to force their content out through their respective outlet orifices according to a predetermined ratio.

30. The device as defined by claim 27, which comprises:
a) a first flexible-wall container containing the component (A);
b) a second flexible-wall container, exterior to the first, and containing the component (B);
c) a plate on which the first and second containers are mounted;
d) a distributing member that is movable with respect to the plate and able to irreversibly switch the device from a first configuration in which the first and second containers are isolated from at least one distribution orifice formed by said distributing member, to a second configuration in which the first and second containers are in communication with said distribution orifice(s).

31. The device as defined by claim 21, comprising a double pump which includes:
a) two reservoirs comprising, respectively, the component (A) or the component (B),
b) two pumps, each mounted on a reservoir,
c) a single push-button that can move axially along a main axis of elongation of the outer casing, and making it possible to activate the simultaneous opening of the two pumps; these two pumps each being connected by virtue of an outlet channel that opens into at least one distribution orifice.

32. The device as defined by claim 21, comprising a double aerosol which includes:
a) two reservoirs comprising at least one propellant and, respectively, the component (A) or the component (B),
b) two aerosol valves, each mounted on a reservoir,
c) a single push-button that can move axially along a main axis of elongation of the outer casing, and making it possible to activate the simultaneous opening of the two pumps; these two valves each being connected by virtue of an outlet channel that opens into at least one distribution orifice.
